(19) United States
(12) Patent Application Publication
(10) Pub. No.: US 2023/0099175 A1
(43) Pub. Date: Mar. 30, 2023

(54) MENSTRUAL DISC WITH RELEASE MECHANISM

(71) Applicant: The Flex Company, Venice, CA (US)

(72) Inventors: Jane Adame, Venice, CA (US); Jonathan Taylor Wiegele, Venice, CA (US); Amanda Lynn Kagiwada, Los Angeles, CA (US); Lauren Schulte Wang, Venice, CA (US)

(21) Appl. No.: 18/061,883

(22) Filed: Dec. 5, 2022

Related U.S. Application Data
(63) Continuation of application No. PCT/US2021/036226, filed on Jun. 7, 2021.
(60) Provisional application No. 63/035,517, filed on Jun. 5, 2020.

(51) Int. Cl. A61F 5/455

(52) U.S. Cl. CPC A61F 5/4553

(57) ABSTRACT

A menstrual disc with a foldable rim and a catchment portion. The rim is configured with a removal mechanism that is positioned attached to the rim such that it does not interfere with the anatomical position of the disc when in use. Accordingly, many embodiments of the disc use a removal mechanism such a filament or tab that is positioned interior to the rim or below the lower surface of the rim.

### CROSS-REFERENCED APPLICATIONS

This application is a continuation of International Application No. PCT/US2021/036226 filed Jun. 7, 2021, which claims priority to U.S. Provisional Pat. Application No. 63/035,517 filed on Jun. 5, 2020, the disclosures of which are included herein by reference in their entirety.

### FIELD OF THE INVENTION

The present invention generally relates to menstrual collection devices, specifically menstrual discs.

### BACKGROUND OF THE INVENTION

A person who menstruates will typically use a tampon or pad to absorb catamenial fluids. Alternatives to tampons and pads exist to collect catamenial fluids such as menstrual cups and/or menstrual discs. Menstrual cups and menstrual discs have been in use and have been known as is described further in U.S. Pat. Nos. 1,891,761 to Goddard, 1,996,242 to Hagedorn, 2,089,113 to Chalmers, 5,827,248 to Crawford, and U.S. Design Pat. No. D323,212 to Crawford and U.S. Pat. No. D837,980. Such devices are commonly used as a replacement to other catamenial devices such as tampons and sanitary napkins.

The typical menstrual cup is a cup like device with a reservoir or catchment area and a rim designed to be inserted into the vaginal canal. Such devices are designed to catch menses. Goddard, Hagedorn, and Chalmers devices are worn in a lower region of the vaginal canal where there are more nerve endings. These menstrual cups form a vacuum seal with the vaginal wall that must be disrupted before the cup is removed.

An alternative class of menstrual cups as is disclosed in U.S. Pat. No. 5,295,984 to Contente or Brazilian Patent Application No. 202019005049 to Azar. Similar to the menstrual cups of Goddard, Hagedorn, and Chalmers, the Contente and Azar devices are designed to be foldable such that they can be inserted into the vaginal canal and then expand once in position. However, both the Contente and Azar devices are designed to sit in the fornix, an anatomical region around the cervix at the top of the vaginal canal where there are fewer nerve endings. Often times this type of menstrual cup is referred to as a menstrual disc. Menstrual cups and discs are often difficult and uncomfortable to remove. Further, removal of the devices can pose health and safety issues as users have to insert their fingers into the vaginal canal. Because menstrual discs sit at the top of the vaginal canal, unlike menstrual cups, they are typically even more difficult to remove and sometimes users have to seek assistance from third parties and health care providers to assist with removal. Because of the high placement of a menstrual disc, individuals with loss of dexterity or grip strength may have even greater difficulty with removal. Menstrual discs may be constructed of materials that are single-use or from materials that are reusable.

Many of the above-mentioned menstrual devices often are designed to accommodate a wide range of users. As such, many fail to consider the various types of anatomy that may be encountered and thus such devices tend to be uncomfortable to many users and difficult to remove. Additionally, in an effort to improve use of such devices, removal methods have been tried which often do not work in the case of menstrual discs because they sit in the fornix, or which are designed in a manner that can cause irritation to delicate vaginal tissue, and therefore fail to appeal to the spectrum of users over time. Therefore, there is a need for an improved removal mechanism for menstrual cups and discs which overcomes one or more of the disadvantages of the prior art.

### BRIEF SUMMARY OF THE INVENTION

Many embodiments are directed to a menstrual disc that has a resilient, foldable, edged rim with an interior surface and an exterior surface and an upper and lower surface. The various surfaces of the rim act to define the cross-sectional shape of the rim. The menstrual disc has a catchment portion that is connected to the resilient, foldable, edged rim and is substantially disposed beneath the lower surface of the rim. The catchment portion has an interior sidewall and an exterior sidewall. The menstrual disc is also configured with a removal mechanism having at least one proximal end connected to the rim and a body portion extending outward from the at least one proximal end, wherein the body portion of the removal mechanism is substantially disposed below the lower surface of the resilient, foldable, edged rim and lateral to the exterior sidewall of the catchment portion.

In other embodiments, the removal mechanism is a filament.

In still other embodiments, the removal mechanism is a molded tab.

In yet other embodiments, the removal mechanism has at least two proximal ends wherein each of the at least two proximal ends are connected to the rim such that the body portion extends between the at least two proximal ends and forms a loop.

In still yet other embodiments, the proximal end(s) of the removal mechanism have a cross-sectional shape that is thinner than that of the body portion.

In other embodiments, the removal mechanism has a textured surface.

In still other embodiments, wherein the at least one proximal end is connected to the interior surface of the rim.

In yet other embodiments, the at least one proximal end is connected to the lower surface of the rim.

In still yet other embodiments, the removal mechanism is removably connected to the rim.

In other embodiments, at least a portion of the removal mechanism extends through a height of the cross-sectional shape of the rim at least once.

In still other embodiments, at least a portion of the removal mechanism extends through a height of the cross-sectional shape of the rim at least twice and forms a connection loop to the body portion of the removal mechanism.

In yet other embodiments, wherein the cross-sectional shape of the resilient, foldable, edged rim is selected from a group consisting of half-domed, triangular, and “U” shaped.

In still yet other embodiments, the cross-sectional shape of the resilient, foldable, edged rim has a leading edge such that the leading edge is chamfered in such a manner that the chamfer is an angled surface directed downward from the upper surface towards the lower surface and between the interior surface and the exterior surface.

In other embodiments, the removal mechanism is formed as part of the resiliently foldable rim in a single molding process.

In still other embodiments, the removal mechanism is molded within the resiliently foldable rim in a secondary molding process.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 is an illustration of a menstrual disc in accordance with known art.

FIG. 2 illustrates a cross sectional view of a menstrual disc in accordance with embodiments of the invention.

FIG. 3 illustrates a rim of a menstrual device in accordance with embodiments of the invention.

FIG. 4 illustrates a menstrual device with a removal mechanism in accordance with embodiments of the invention.

FIG. 5 illustrates a rim of a menstrual device in accordance with embodiments of the invention.

FIG. 6 illustrates a menstrual device removal mechanism in accordance with embodiments of the invention.

FIGS. 7A and 7B illustrate a menstrual device with a removal mechanism in accordance with embodiments of the invention.

FIG. 8 illustrates a menstrual device in accordance with embodiments of the invention.

FIG. 9A illustrates cross sectional views of rim designs in accordance with embodiments of the invention.

FIG. 9B illustrates a cross sectional view of a rim design in accordance with embodiments of the invention.

### DETAILED DESCRIPTION OF THE INVENTION

Turning now to the drawings, menstrual devices with removal mechanisms are provided. In accordance with many embodiments, menstrual discs may incorporate a removal mechanism that is connected to a portion of a rim element such that the removal mechanism does not negatively interact with the anatomy of a user. Various embodiments of a menstrual disc incorporate a tab or a pull mechanism that is connected to an inside or underside portion of the rim where the catchment portion of the device is disposed on the interior of the rim yet exterior to the pull mechanism. Some embodiments may incorporate a filament that is integrated into the rim.

Menstrual discs, as previously described, have been in use and are well known in the field of menstrual devices. However, known menstrual discs can have many disadvantages. The ease of insertion and removal can present certain difficulties that may lead users to resort to less ideal methods of collection. For example, known discs generally lack a function to aid in the removal. Most discs require the user to grasp a portion of the rim of the disc and pull remove the device. This traditional method of removal is often messy and can be uncomfortable for some users.

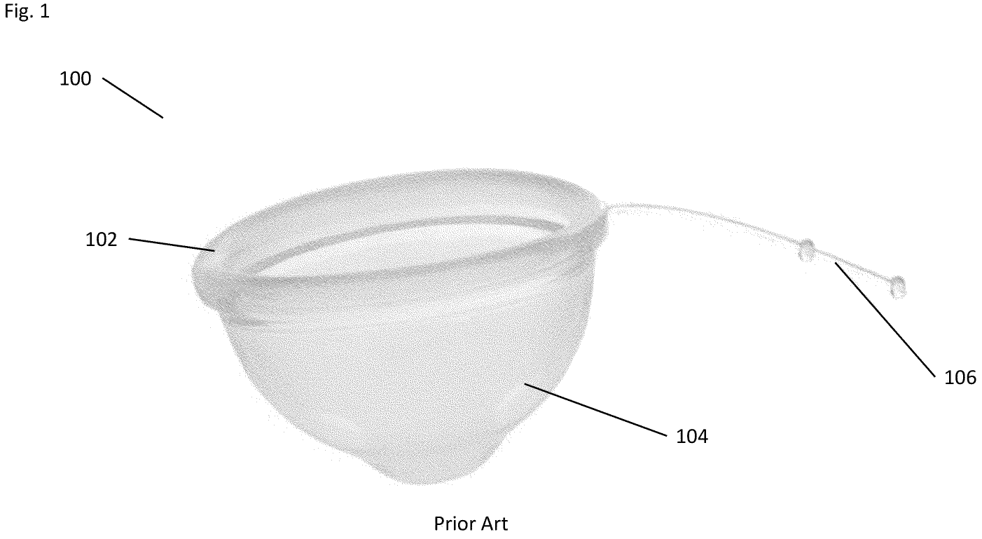

In some discs, removal devices have been integrated. However, the integration of such devices tend to pose a potential for discomfort during use. For example, FIG. 1 illustrates a menstrual disc with a removal string in according to the prior art of Azar. The menstrual disc 100 has a rim 102 with a catchment portion 104. In some prior art discs, the rim 102 is configured with a tab 106 device that is attached to the outside portion or front face of the rim 102 as describe in Azar. The tab 106 is designed to rest in the vaginal canal of the user and provide the user with a method of removal that does not involve gripping the rim 102 of the disc 100. Although such removal strings 106 can help in the removal of the device 100, the particular position of such strings or tabs can present potential issues with use. For example, given the anatomical position in which the disc must sit to be effective, the string or tab 106 positioned on the front face would necessarily place the string or tab 106 between the rim 102 and the vaginal walls of the user. Accordingly, such interaction could result in undue comfort for the user.

Note that a menstrual disc has a relatively robust resilient rim that opens around the cervix in the vaginal fornix. This is quite advantageous with regard to comfort as the fornix is relatively wide as compared to the main portion of the vaginal cavity. The robust nature of the rim keeps the disc secured and prevents fluids from leaking. A user is thus freed from the toxic shock danger of tampons and the clutter of sanitary napkins and may even engage in intercourse without leakage of menses. During insertion, the rim is folded but as it nears the fornix the rim expands into position. However, the comfort and fit of a conventional menstrual disc comes at a cost of a potentially difficult removal. The relatively robust rim of the menstrual disc needs to be deformed so that disc may fold and be retrieved from the vagina. Depending upon a woman’s anatomy, it may be difficult for a user to extend a finger sufficiently within the vagina to be able to deform the resilient rim and begin retrieval.

To aid in removal, it is known to attach a cord or lead to the rim as discussed with regard to FIG. 1. But this cord then protrudes from the rim and may lead to user discomfort. To solve these removal problems yet retain the comfort and fit of a menstrual disc, the rim may be edged as disclosed herein so as to overlap a catchment portion of the menstrual disc. Due to this overlapping of the rim onto the catchment portion, an inner surface of the rim faces an exterior surface of a catchment portion of the menstrual disc. A potential cavity thus exists between the inner surface of the rim and the exterior surface of the catchment portion. A removal mechanism may then be placed within this cavity so as to attach to the inner surface (and/or bottom surface) of the rim. This is remarkably advantageous in that the removal mechanism may then have a distal extension or body portion that a user may readily grasp despite any vagaries in anatomy. The overlapping of the rim onto the catchment portion shields the user from sensing the removal mechanism. A user may thus have the comfort and health advantages of a menstrual disc without any removal difficulties. Some example embodiments will now be discussed in more detail.

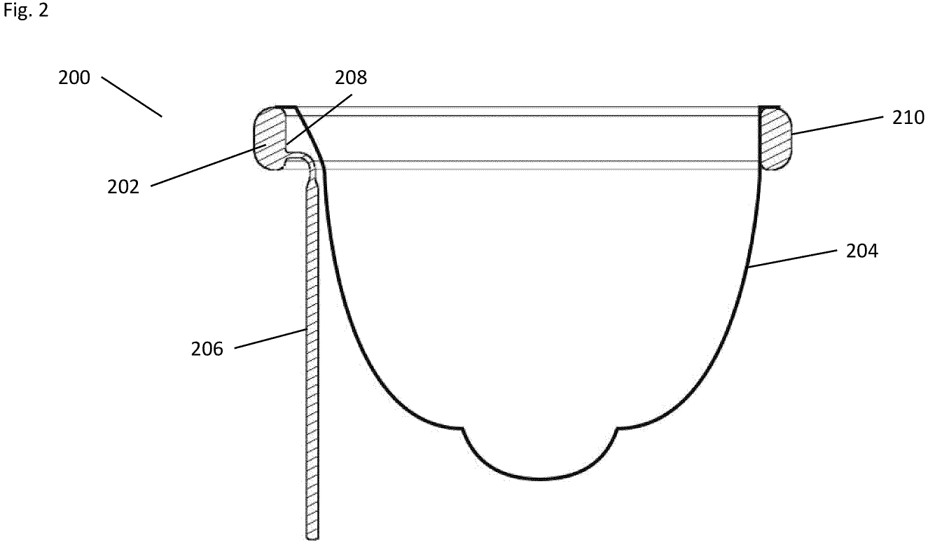

FIG. 2 illustrates a cross sectional view of a menstrual disc 200 with a rim 202 and a catchment 204 portion. The rim, in numerous embodiments, may incorporate a removal mechanism 206 that is connected to an interior portion 208 of the rim 202. The removal mechanism 206 can take on any number of forms such as a thinner tab like device as seen in FIG. 2. In numerous embodiments, the catchment portion 204 is connected to the rim 202 such that it is disposed on the interior portion 208 of the rim 202. As can be appreciated the shape of the rim 202 can be a circular ring that has an exterior surface 210 as well as the interior portion 208. Likewise, it can be readily understood that the exterior surface 210 is the portion of the disc 200 that interacts with the vaginal walls of the user. In various embodiments, the removal tab 202 is disposed on the interior portion 208 of the rim and rests between the catchment portion 204 and the rim itself. Such position allows for the removal mechanism 206 to rest below the rim 202 and remain in a position that would not interfere with the rim 202 and the vaginal walls of the user. This allows for maximum connection between the exterior surface 210 of the rim 202 and the vaginal walls of the user while providing a mechanism to help in the removal of the menstrual disc 200. Accordingly, many such embodiments allow for the easy removal of the disc while simultaneously avoiding the potential for discomfort during use.

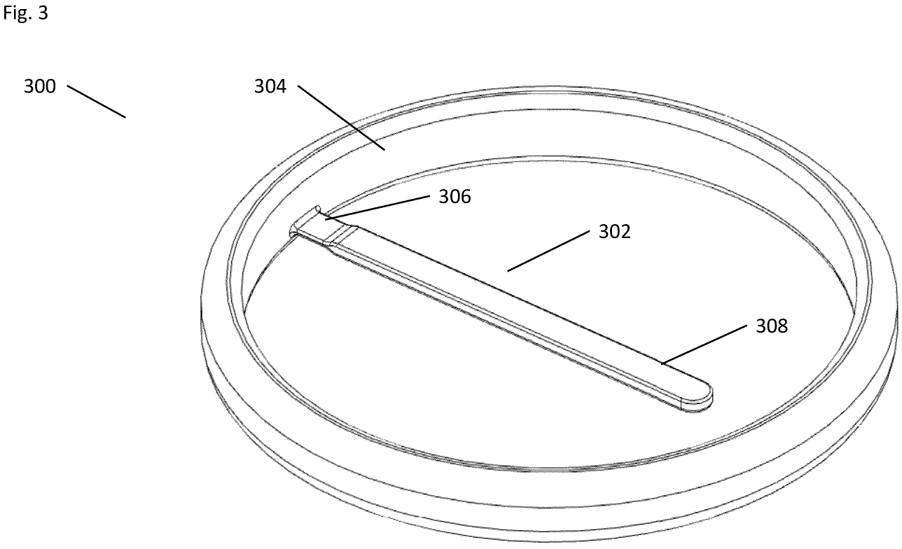

The use of a removal mechanism as illustrated herein can provide a number of advantages over other menstrual discs that can be difficult to remove. In accordance with numerous embodiments, the removal mechanism can take on any number of shapes, sizes, and be made of any type of material. Additionally, the removal mechanism can be placed in any desirable location such that it does not interfere with the vaginal walls of the user. In some embodiments, the removal mechanism, as illustrated in FIGS. 2 and 3, can be formed to be an appendage of the rim itself.

Many embodiments of a menstrual disc may incorporate a removal tab that varies in size and shape. For example, numerous embodiments may have a removal tab that is connected to an inside portion of disc that changes in cross sectional dimension or may be made of a number of different materials. FIG. 3 illustrates a rim 300 for use in a menstrual device that incorporates a removal tab 302 having a variable cross-sectional dimension in accordance with embodiments of the invention. In various embodiments, the rim 300 may be configured with a removal tab 302 that is connected to an inside portion 304 of the rim 300. In accordance with many embodiments, the removal tab 302 may be an elongated member with a proximal end 306 and a distal end 308. The distal end 308, in accordance with some embodiments, may have a cross sectional width that is wider or thicker than that of the proximal end 306. It can be appreciated that the length and cross-sectional design of the removal tab 302 can vary with many embodiments. Furthermore, it can be appreciated that the length and cross-sectional design of the proximal and distal portions of the tab can vary and the tab illustrated in FIG. 3 is merely an exemplary embodiment of a rim 300 with a removal tab 302.

FIG. 3 further helps to illustrate the various methods of manufacture that can be used in producing a menstrual disc with a removal tab 302 in accordance with embodiments of the invention. Specifically, FIG. 3 illustrates a stand-alone rim 300 that can be manufactured in any number of methods including molding. Accordingly, the tab 302 may remain elongated and disposed entirely within the interior portion 304 of the rim 300. However, as illustrated in FIG. 2, the menstrual disc requires the use of a catchment portion, which can be welded after the molding of the rim with the removal tab. This can allow for the removal tab 302 to remain on the interior portion 310 of the rim and then rest below the rim when in use, as illustrated in FIG. 2. Additionally, given that some embodiments may incorporate a two-part manufacturing method; it can be appreciated that the various portions (i.e. rim, catchment, and tab) can be made from the same or different materials. It can further be appreciated that many embodiments may use one or more durometer type materials in the manufacture of the different portions of the disc.

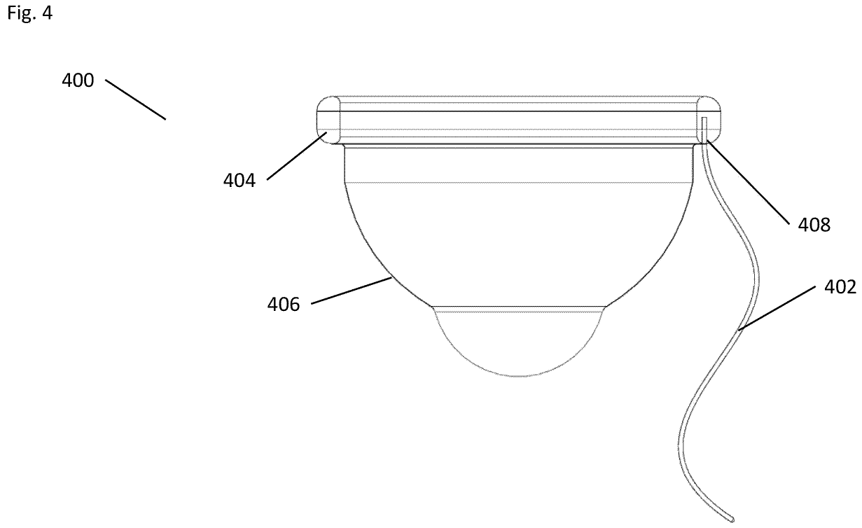

Although many embodiments of a menstrual disc may incorporate a removal tab, numerous embodiments may incorporate alternative removal mechanisms. For example, FIG. 4 illustrates a menstrual disc 400 with a removal filament 402 in accordance with embodiments of the invention. The menstrual disc 400, similar to traditional menstrual discs may incorporate a rim 404 and a catchment portion 406. In order to improve the overall comfort and use of the menstrual disc 400, various embodiments may utilize a removal filament 402 that is positioned within the rim 404. The filament 402 can be molded within the rim 404 during the manufacturing process such that it is positioned below the bottom portion 408 of the rim. The filament 402, in accordance with various embodiments, can be of any desirable length and can be manufactured of any desirable material. For example, some filaments may be made of polyethylene. Likewise, many filaments may be made of the same or different material from that of the rim and/or catchment portions. Moreover, it can be appreciated that many embodiments may use one or more durometer type materials in the manufacture of the different portions of the disc and/or the removal mechanism.

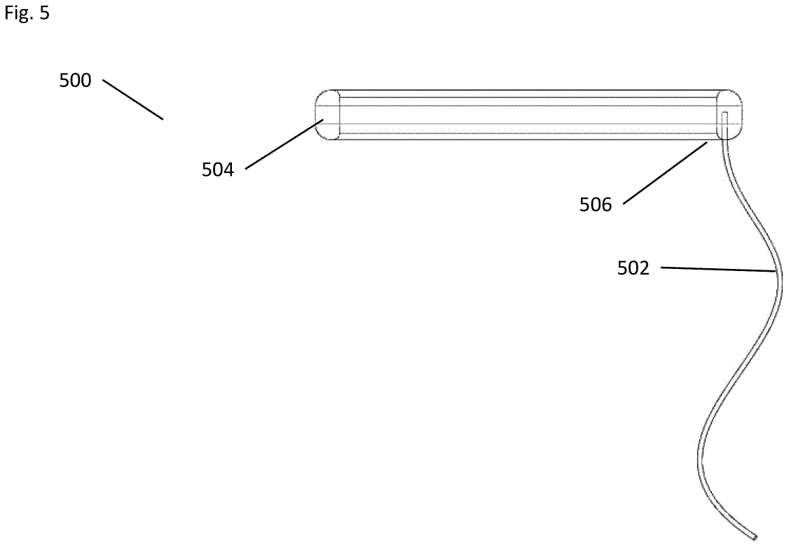

Similar to FIG. 3, FIG. 5 illustrates a rim 500 that is separate from a catchment portion. Likewise, as described in relation to FIG. 3, the rim 500 can be manufactured separately in a different manufacturing step from that of the catchment portion. In many such embodiments, the rim 500 may be configured with a removal mechanism 502 that is molded directly into the inner portion 504 of the rim and is configure to rest below the bottom portion 506 of the rim 500 similar that that illustrated in FIG. 4. Such rims 500 can alternatively be secondarily molded or molded in a second step to incorporate a catchment portion that does not interfere with the positioning of the removal filament 502.

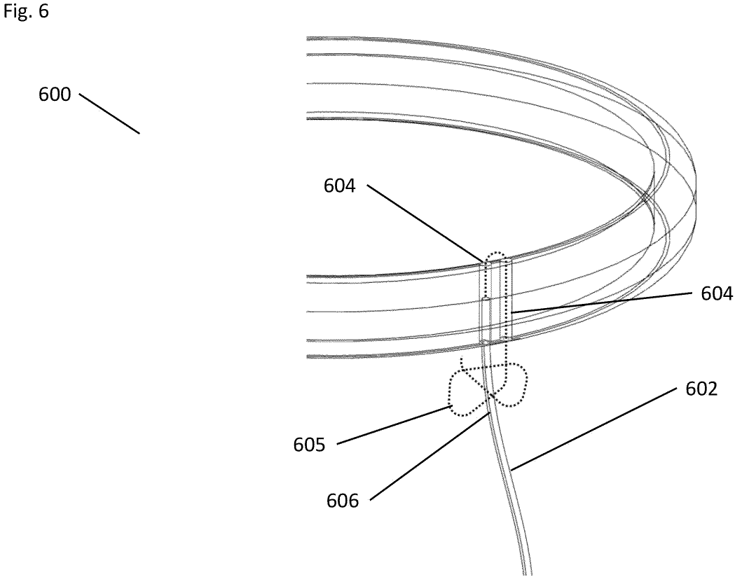

Alternatively, various embodiments may incorporate a removal mechanism that can be installed or attached after the formation of the rim and/or catchment portion. For example, FIG. 6 illustrates a rim 600 of a menstrual disc with a removal mechanism 602 in the form of a filament that is removable from the disc. The removable filament or removal mechanism can extend through the height of the rim in a number of ways. For example, as illustrated, a portion of the removal mechanism can extend through two different holes forming a connecting loop 605 within the body of the rim and reconnecting with a body portion 606 of the removal mechanism. Other embodiments may only extend through the rim through a single hole where the removal mechanism can be inserted and/or removed. As can be appreciated the position of the removal filament 602 or any removal device can be a determining factor in the overall operation of the disc. Thus, numerous embodiments aim to position the removal filament 602 or tab such that it is not positioned on an outside portion of the rim 600.

The rim 600, in accordance with many embodiments, may be manufactured with one or more connection ports 604. The connection ports 604 can be a series of holes or a single hole located within the interior portion of the rim 600. The connection ports 604 can allow for any number of removal filaments and/or tabs 602 to be connected to the rim 600 through a number of connections means such as tying, subsequent bonding through molding, or any number of techniques. This can allow for the rim 600 to be more versatile such that any number of different removal mechanisms such as longer or textured can be used for any number of different users. The adaptability of a rim 600 to accommodate a number of different types of removal mechanisms 602 can be preferable in some embodiments where the manufacturing may not allow the two components to be co-molded or manufactured in sequence. Additionally, many embodiments may incorporate the use of different materials that are not compatible during the manufacturing process. Therefore, having a rim 600 with the ability to receive any type of removal mechanism can be preferable on various embodiments.

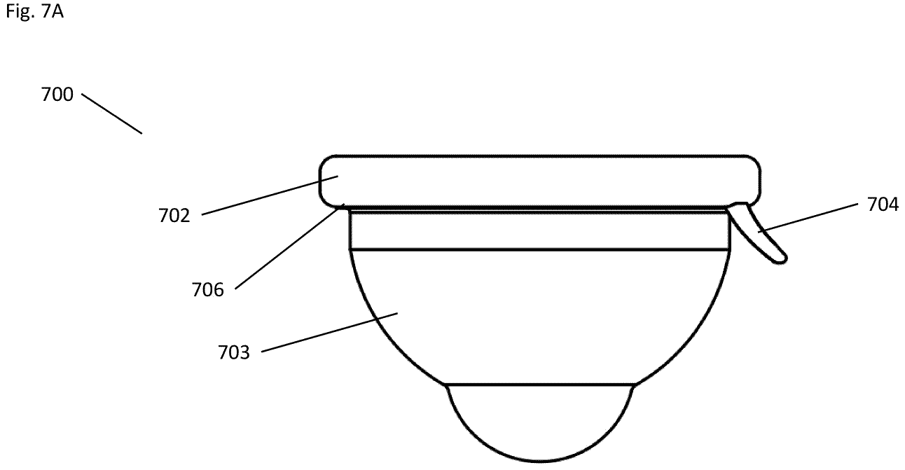

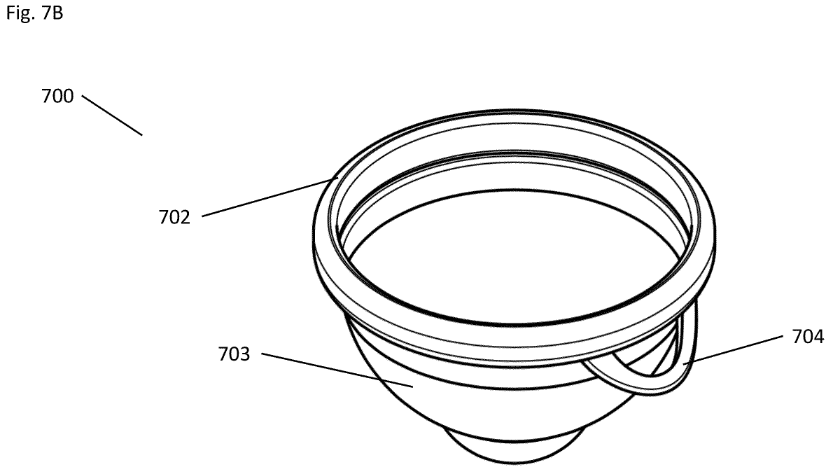

Although various embodiments illustrated herein describe a removal mechanism in the form of a tab or filament that is an elongated body extending below the bottom portion of the rim, numerous embodiments may utilize an alternative form of a removal mechanism that is similarly situated with respect to the rim. For example, FIGS. 7A and 7B illustrate an embodiment of a menstrual device 700 with a rim 702 and a catchment portion 703 disposed beneath the rim 702. The device 700 can be configured with a removal mechanism 704 that is positioned below the bottom portion 706 of the rim and is connected to the rim in at least two separate locations forming a loop. The loop design can provide the ability for the disc 700 to be easily removed from a person who might have issues with gripping a small thin tab. As can be appreciated, some embodiments may have one or more loops that can allow for the easy removal of the disc.

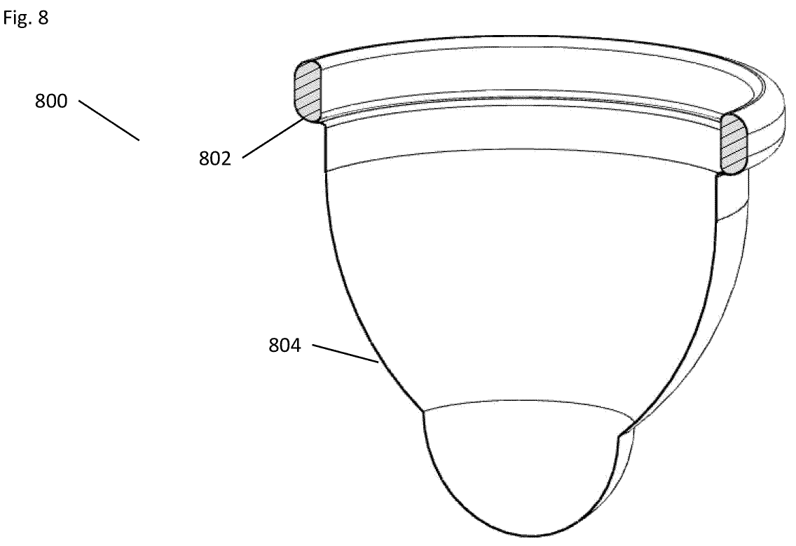

As has been described throughout, many embodiments can vary in composition of components and overall design to allow for increased comfort for the users. As such many embodiments may utilize a removal mechanism that has additional features that can improve the ability for the user to grip the mechanism. For example, some embodiments may have texturing or as described previously different cross-sectional designs that can help with the gripping of the mechanism. Additionally, it can be appreciated that menstrual discs may have different catchment designs to allow for increased comfort by allowing for longer use and or use during heavier cycles. FIG. 8 illustrates a menstrual disc 800 with a rim portion 802 and a catchment portion 704 in accordance with some embodiments. The catchment portion 804 may be more elongated than a traditional disc to provide for an improved ability to collect during heavier cycles. Such embodiments can be desirable for a number of users to prevent the need to remove the disc more often. While a specific embodiment of a menstrual disc 800 with an elongated catchment portion 804 is illustrated, it can be appreciated that numerous embodiments may have elongated catchment portions 804 to varying degrees. It further can be appreciated, that an elongated catchment may itself serve as a removal mechanism aiding in the locating and applying a downward force on the rim portion of the device. Furthermore, it can be appreciated that such embodiments can be configured with a removal mechanism, not shown, similar to those described in FIGS. 2-7.

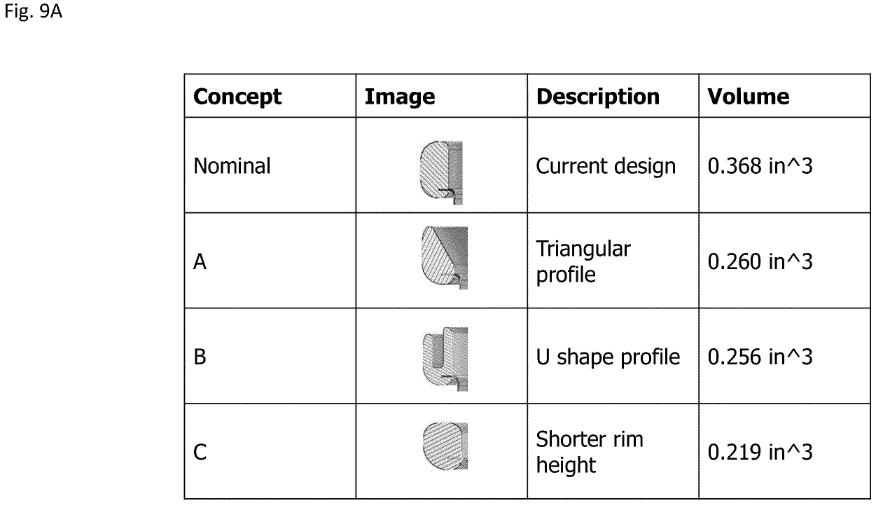

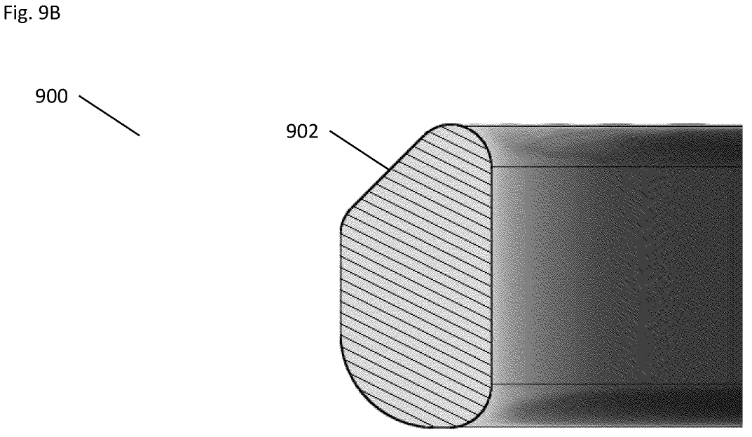

In addition to the numerous configurations between the various menstrual disc components, it can be appreciated that some embodiments of a menstrual disc with a removal mechanism may incorporate rims with one or more cross sectional designs. For example, FIG. 9A illustrates a table with various cross sections of rims and the associated volume of material. Accordingly, it can be seen that the various cross sections can reduce the material used. This indicates that the cross section can be optimized for improved environmental impact as well as usability. Furthermore, the design of the rim can improve the overall function and insertion capabilities of the menstrual device. For example, FIG. 9B illustrates a cross-sectional view of a rim 900 with a chamfered leading edge 902. The chamfered leading edge 902 can help to redistribute the forces applied to the rim during insertion. As such the positioning of the menstrual disc can be improved because the positioning angle of the disc can be directed or controlled by the contoured or chamfered leading edge. Other embodiments can have a cross-sectional shape that can provide other advantages such as a dome shape, triangular shape, U-shaped, or any other suitable shape for manufacturing and use. Furthermore, in some embodiments the geometric dimensions can vary. In some embodiments, the rim diameter can be between 2ʺ and 3ʺ. In other embodiments, the height of the rim may be between 0.25ʺ and 1ʺ. Likewise, the width of the rim may be between 0.25ʺ and 1ʺ, in accordance with embodiments.

### DOCTRINE OF EQUIVALENTS

As can be inferred from the above discussion, the above-mentioned concepts can be implemented in a variety of arrangements in accordance with embodiments of the invention. Specifically, menstrual discs with a removal mechanism that does not interfere with the anatomical position of the disc when in use. Achieving such functionality, according to embodiments, involves the implementation of special arrangements/designs between subsystems described above, and their equivalents.

Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A menstrual disc comprising:
a catchment portion that has an interior sidewall and an exterior sidewall;
a resilient rim connected to the catchment portion and having an interior surface facing the exterior sidewall of the catchment portion;
a resilient rim having an outer edge
a removal mechanism connected to the inner surface of the resilient rim.

2. The menstrual disc of claim 1, wherein the removal mechanism is a filament.

3. The menstrual disc of claim 1, wherein the removal mechanism is a molded tab.

4. The menstrual disc of claim 1, wherein the removal mechanism forms a loop connected to two points of the inner surface of the rim.

5. The menstrual disc of claim 4, wherein the removal mechanism is a filament.

6. The menstrual disc of claim 4, wherein the removal mechanism is a molded tab.

7. The menstrual disc of claim 4, wherein each of the at least two proximal ends have a cross-sectional shape that is thinner than that of the body portion.

8. The menstrual disc of claim 1, wherein the removal mechanism has a textured surface.

9. The menstrual disc of claim 1, wherein a first portion of the removal mechanism has a cross-sectional shape that is thinner than that of a second portion of the removal mechanism that is connected to the inner surface of the rim.

10. The menstrual disc of claim 1, wherein the removal mechanism also connects to a portion of a bottom surface of the rim.

11. A menstrual disc comprising:
a catchment portion that has an interior sidewall and an exterior sidewall;
a resilient rim connected to the catchment portion and having an interior surface facing the exterior sidewall of the catchment portion, an exterior surface facing away from the exterior sidewall of the catchment portion, and a bottom surface between the interior surface and the exterior surface;
a resilient rim having an outer edge
a removal mechanism forming a loop having a first end connected to at least one of the bottom surface and the inner surface and a second end connected to at least one of the bottom surface and the inner surface.

12. The menstrual disc of claim 11, wherein a cross-sectional shape of the resilient rim is selected from a group consisting of half-domed, triangular, and “U” shaped.

13. The menstrual disc of claim 11, wherein a top surface of the rim is chamfered at an intersection of the top surface and the exterior surface.

14. The menstrual disc of claim 11, wherein the removal mechanism is formed with the resilient rim in a single molding process.

15. The menstrual disc of claim 11, wherein the removal mechanism is molded within the resilient rim in a secondary molding process.
